(19) United States
(12) Patent Application Publication
(10) Pub. No.: US 2022/0193060 A1
(43) Pub. Date: Jun. 23, 2022

(54) Inhibition of IL-1 Induced Inflammation

(71) Applicant: THIOLAB, LLC., Berkeley, CA (US)

(72) Inventors: Davide R. Grassetti, Jamestown, CA (US); Camillo Moro, Padova (IT); Richard A. Grassetti, Berkeley, CA (US)

(21) Appl. No.: 17/605,447

(22) PCT Filed: May 1, 2020
(86) PCT No.: PCT/US2020/030974

§ 371 Date: Oct. 21, 2021

Related U.S. Application Data
(60) Provisional application No. 62/842,081, filed on May 2, 2019.
(60) Provisional application No. 63/010,553, filed on Apr. 15, 2020.

(51) Int. Cl. A61K 31/455; A61K 31/44; A61K 31/506; A61K 31/427; A61P 37/06

(52) U.S. Cl. CPC A61K 31/455; A61K 31/44; A61P 37/06; A61K 31/427; A61K 31/506

(57) ABSTRACT

This invention provides methods of treatment for a variety of inflammation pathologies using thione-forming disulfides (TFDs). The treatments are particularly effective in the context of inflammations primarily due to excessive activity of innate immune system components, macrophage activity and inflammation arising from excessive production of IL-1. TFD inhibition of both IL-1 and IL-6 can reduce the detrimental effects of the cytokine storms known to increase morbidity and mortality caused by certain infections, such as COVID-19 viral pneumonia.

### FIELD OF THE INVENTION

This invention is in the general field of immunology. More specifically, the invention relates to methods of treating inflammation or inflammatory disease by using thione-forming disulfides (TFDs), including treatments with 6,6′-dithiodinicotinic acid (CPDS). Certain aspects the invention are concerned with inhibition of overactive cytokines, such as IL-1β and IL-6.

### BACKGROUND OF THE INVENTION

Inflammation has traditionally been divided into two types of responses: acute and chronic. Acute inflammatory responses are usually rapid and short-lived (minutes to days). Acute inflammation can be characterized by accumulation of fluid, plasma proteins, and neutrophilic leukocytes. Acute inflammation can build to a cytokine storm, in certain infections, increasing the chances of permanent tissue damage or death. Chronic inflammation is longer-lived and can be characterized by an influx of lymphocytes, macrophages, and fibroblast growth. See, for example, Fundamental Immunology (W. E. Paul, ed., 1999). Sources of inflammation can be based on specific immune responses (immune memory) and/or inflammation based on non-specific (innate) immune responses.

A variety of different enzymes and cytokines have been investigated to determine their roles in inflammation. One enzyme is poly (ADP-ribose) synthetase, whose role in cellular injury and inflammation has been reported in Szabo et al. Eur. J. Pharmocol. 350 (1): 1-19 (1998). Methods of treating inflammation by using inhibitors of this enzyme are disclosed in U.S. Pat. No. 5,908,861. Another enzyme which has been investigated for its role in inflammation is cyclooxygenase-Z (COX-2). Treatments for inflammation which involve COX-2 are disclosed in several patent applications, including: U.S. Pat. Nos. 6,264,995; 6,077,869; and 6,472,433. Additional insights in the intervening years strongly suggest that modulators of the immune response, including but not restricted to cytokines, can also have a profound regulatory effect on inflammation and has been the target for novel therapeutic agents directed towards the to modulate inflammatory conditions (see below).

Many methods of treating inflammation have been disclosed. See, for example, e.g., U.S. Pat. Nos. 5,244,899; 5,071,876; 5,908,620; 5,906,815; 6,271,253; 6,265,374; and 6,248,790. Methods of treating inflammation using compounds which contain sulfur have been disclosed. See, for example, U.S. Pat. Nos. 6,284,918; 6,191,170; 6,117,889, and 6,110,922.

Many of the analgesics have undesirable side effects or are toxic in certain individuals at required doses. The available analgesics work on one kind of pain, but less well for others. Inflammation can result from any number of tissue interactions, e.g., with toxins, antigens, or microbes. No anti-inflammatory or analgesic safely addresses all the needs of patients.

IL-1β is one of the major cytokines implicated in the pathogenesis of many inflammatory-associated diseases. Recent studies demonstrate that IL-1β is activated through inflammasomes, which are formed upon recognition of danger signals by the immune system. IL-113 is, therefore, becoming a focus for the development of new anti-inflammatory drug products. Current issued patents mainly covered the methods and the use of four types of IL-1β blockade compounds, namely anti-IL-1β antibody, IL-1 receptor antagonists such as sIL-1Ra and icIL-1Ra, and IL1 trap. Two agents, Rilonacept and canakinumab were approved by the US FDA and others are in trial, in which beneficial results have been reported. However, these drugs are expensive and complicated to manufacture and administer, and may have detrimental side-effects.

IL-6 is an important cytokine in recruiting other cytokines during an infection. This recruitment can be overdone, resulting in a possibly damaging self-perpetuating cytokine storm. IL-6 also plays an important role in other inflammatory processes, such as in rheumatoid arthritis. It is notable that the Covid-19 pandemic has caused a significant percent of those infected to experience major detrimental symptoms due to an excessive cytokine response. This “cytokine storm” has initially been associated with over production of IL-6, possibly along with IL-1, TNF, and interferon-γ. See, e.g., Giovanni Monteleone correspondence of Lancet Rheumatology, Apr. 6, 2020. Typically, IL-6 is clinically inhibited using a monoclonal antibody, e.g., Tocilizumab™. However, monoclonal antibodies do not diffuse or penetrate well through certain infections or tissues. Further, such antibody-based therapies are expensive and can open the patient up to new or reinvigorated bacterial, viral, and/or fungal infections.

In view of the above, additional anti-inflammatories are needed. Benefits also could be realized from anti-inflammatories with low toxicity. It would be useful to have additional anti-inflammatories taking advantage of a new mode of action, e.g., for use alone or in combination with companion analgesic and/or anti-inflammatory drugs. With regard to IL-1β and IL-6, in particular, we believe benefits can be substantial from a relatively non-toxic small molecule inhibiter. The present invention provides these and other features that will be apparent upon review of the following.

### SUMMARY OF THE INVENTION

Provided herein are methods for treating inflammation using thione-forming disulfides. Accordingly, in one aspect, a method for treating inflammation in an individual comprises administering an effective amount of a thione-forming disulfide (TFD) to an individual to reduce pathologies of inflammation. In certain embodiments, the inflammation addressed by the current treatments can be those pathologic manifestations of inflammation associated with innate immunity responses, particularly those associated with the cytokines IL-1 (α and/or β), and IL-6.

Methods of treating an individual are provided (e.g., with TFDs, or CPDS), wherein the disease state is associated with an inflammation. For example, the disease state can be arthritis, Covid-19 infection, breast hyperplasia, psoriasis, edema, colonic polyps, innate immunity over-reaction, disease states associated with a variety of immune effector cells, e.g., excess macrophage or reticuloendothelial system activity, and/or the like. For example, CPDS can be used to treat sickle cell disease, or diabetes inflammation. In addition, the TFDs can be used to treat, e.g., cryopyrin-associated periodic syndromes (CAPS) including familial cold auto-inflammatory syndrome (FCAS), systemic juvenile idiopathic arthritis (SJIA), neonatal-onset multisystem inflammatory disease (NOMID), rheumatoid arthritis, cancer, atherosclerosis, diabetes, infection by viruses and microbes, systemic lupus erythrematosus, schizophrenia, depression, major depressive disorder, Alzheimer's disease, sepsis, and acute respiratory distress syndrome and Muckle-Wells syndrome (MWS). The methods of treatment can provide benefits in fighting cancers, particularly those induced or augmented by inflammations associated with IL-1 activity.

In yet another aspect, a method is provided for treating inflammation in an individual using the thione-forming disulfide 6,6′-dithiodinicotinic acid (CPDS). Typically, the thione-forming disulfide is administered orally. Optionally, the thione-forming disulfide can be administered topically, e.g., wherein the inflammation is due to topical contact with an inflammatory agent, such as poison oak, poison ivy, or poison sumac. In yet another aspect, a method for treating inflammation in an individual is provided, in which the inflammation is mediated by arachidonic acid.

In yet another aspect, a method for providing analgesic relief in an individual comprises administering an effective amount of thione-forming disulfides to an individual in need of analgesic relief. In yet another aspect, a pharmaceutical composition of thione-forming disulfides is provided for reducing inflammation. In particular, the pharmaceutical composition of thione-forming disulfides for reducing inflammation can be CPDS.

In certain embodiments, the methods of treating disease states can include identifying a patient with an IL-1β and/or IL-6 inflammation pathology clinical indication and administering an effective dose of a thione-forming disulfide to the patient. At the effective dose, there are typically no substantial toxic side effects, while IL-1β and IL-6 are inhibited in the patient and the inflammation pathology is reduced in the patient. In such embodiments, the clinical indication can be, e.g., an auto-inflammatory disease, inflammation due to a viral (e.g., Coronavirus) infection, an inflammation due to an innate immune response, an inflammation response to a lipopolysaccharide, an inflammation due to activation of macrophages, and/or the like, among pathologic inflammations due to over-expression of IL-1β and/or IL-6. The patient suffering from excessive IL-1β is typically not a cancer patient. Alternately, the CPDS is administered to reduce inflammation that can induce a cancer or create conditions conducive to cancer growth or malignancy. In addition, the TFDs, such as CPDS, can be used to treat, e.g., cryopyrin-associated periodic syndromes (CAPS) including familial cold auto-inflammatory syndrome (FCAS), systemic juvenile idiopathic arthritis (SJIA), neonatal-onset multisystem inflammatory disease (NOMID), diabetes, multiple sclerosis, sickle cell, and Muckle-Wells syndrome (MWS).

In the methods of inhibiting IL-1β and/or IL-6, the thione-forming disulfide can be selected from 6,6′-dithiodinicotinic acid (CPDS), 6,6′-dithiodinicotinic acid diethyl ester, 4-carboxypyrimidine-2-disulfide, diethyl 2,2′-dithiobis-(4-thiazole carboxylate), and 2,2′dithiobis-isonicotinic acid, and/or the like. The administered thione-forming disulfide dose can typically range from 10 μg/kg to 5 g/kg body weight, e.g., in human patients. In preferred embodiments, the patient is a human and the thione-forming disulfide dose ranges from 50 mg to about 1200 mg per day, or about 800 mg per day. Administration can be by oral administration, topical administration, or intravenous administration. The selected dose preferably reduces the IL-1β and/or IL-6 expression in the patient by at least 50 to 95%, or by at least 10%, 20%, 30%, 40%, 50%, 60%, 70%, 80%, 85%, 90%, 95%, or more. Effective doses at these levels typically do not induce substantial toxic side effects in the patient.

The methods of treatment are further directed to mitigating morbidity and mortality caused by viral pneumonia. For example, a method of treating a viral pneumonia can include identifying a patient with viral pneumonia and administering a dose of thione-forming disulfide to the patient. The dose is at a level effective in reducing levels of IL-6 in the patient. In this way, inflammation is reduced in the patient's lungs due to inhibition of IL-6.

In certain embodiments, the viral pneumonia is caused by a Coronavirus, such as MERS, SARS or SARS-CoV-2 (causing COVID-19 disease). A preferred thione-forming disulfide is CPDS. A preferred CPDS dose is 30 mg/kg or more, e.g., orally or by intravenous injection. In one embodiment, the dose provides a concentration of at least 30 μM of CPDS in the patient's blood.

In a specific embodiment, the method treats a COVID-19 viral pneumonia by identifying a patient with COVID-19 viral pneumonia and administering CPDS to the patient with a blood oxygen of less than 90%. The administered dose is at least 30 mg/kg, thereby substantially inhibiting IL-6 and reducing inflammation in the lungs of the patient.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” can include plural referents unless the content clearly dictates otherwise. Thus, for example, reference to “a TFD” can include a combination of two or more TFDs; reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein, “inflammation” refers to the complex biological response of body tissues to stimuli, such as pathogens, allergens, foreign bodies, toxins, damaged cells, or other irritants, as known in the art. Inflammation can provide benefits in fighting infection and creating an environment for rejuvenation of damaged tissue structures. Inflammation is generally a protective response involving, e.g., interactions of blood vessels, immune cells, and cytokines. Inflammation can be a localized phenomenon wherein part of the body becomes reddened, swollen, hot, and often painful, especially as a reaction to injury or infection. Inflammation can also be a more generalized or chronic condition, e.g., due to oxidative stress, elevated cytokine levels, or generalized pyrogens spread diffusely throughout the body, and/or the like. Chronic inflammation can cause tissue damage leading to, e.g., fibrosis, heart disease, cancer, Alzheimer's disease, and/or the like. Different inflammation episodes can result from excess activity from various immune system actors, such as, e.g., innate immunity cells, humoral (e.g., antibody), immune memory cells (e.g., β-lymphocytes or T-lymphocytes), neutrophils, macrophages, other granulocytes (e.g., eosinophils), complement cascade, cytokines, chemokines, and/or the like. In the context of inflammation not due to specific antigen recognition, the cytokines IL-1 and IL-6 can be major players in initiating and/or prolonging the non-specific inflammation response, e.g., such as that associated with certain innate immune reactions, immune responses associated with macrophage or reticuloendothelial (RES) activity, or additional immune responses other than those caused directly by lymphocytes, including those that function via soluble mediators including specific cytokines. That is, there are indications wherein excessive IL-1 (α and/or β) or IL-6 activity is causing inflammation pathologies in a patient that are not the result of immune memory (e.g., lymphocyte) activation. A severe form of inflammation can be a “cytokine storm”, such as that associated with Covid-19 infection.

An “effective amount” of an agent is an amount sufficient to effect beneficial or desired results including clinical results. An effective amount can be administered in one or more administrations by various routes of administration. An effective amount of TFDs described herein in one embodiment is an amount sufficient for a desired amelioration or palliation of inflammatory responses. The inflammatory responses can involve, for example, increasing or decreasing certain populations of cells (e.g., T cells, NK cells, B cells, macrophages, granulocytes, etc.), increasing amounts of pro-inflammatory cytokines (e.g., IL-1, IL-4, IL-6, IL-8, IL-12, TNF-α, TNF-β, and/or IFN-γ) released or produced, increasing or decreasing functional activity of immune cells, or activating or repressing immune cells (e.g., switch from Gₒ to G₁ M₂ to M₁, or switch on/off signaling cascade). In terms of treatment; an “effective amount” of TFDs described herein is an amount sufficient to palliate, ameliorate, stabilize, reverse, slow or delay progression of a disease. In addition, it also includes an amount sufficient to palliate, ameliorate, stabilize, reverse, slow or delay progression of an uncomfortable or undesirable physiological state suffered by an individual. An effective amount of a TFD can be an amount that reduces inflammation caused by innate (e.g., non-immune memory) responses.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

As used herein, “treatment” is an approach for obtaining beneficial or desired results in a patient, including and preferably clinical results.

An “individual” is a vertebrate, preferably a mammal, more preferably, a human Mammals include, but are not limited to, farm animals, sport animals, pets; primates, mice, and rats.

As used herein, “essentially pure” and “substantially pure” CPDS or thione-forming disulfides (TFDs) refers to a composition which is comprised at least 80% CPDS or TFDs, more preferably at least 85%, more preferably at least 85%, more preferably at least 90% CPDS or TFDs, more preferably at least 95%, more preferably at least 98%, and even more preferably at least 99.5%.

### BRIEF DESCRIPTION OF THE DRAWINGS

FIG. 1 presents a chart comparing IL-1β levels for a dosage series of CPDS in the presence of IFN-γ. These data are compared to lactoferrin and clobetasol controls. See Appendix for procedures and data.

FIG. 2 presents a chart comparing IL-1β levels for a dosage series of CPDS in the presence of LPS.

FIG. 3 presents a chart comparing IL-6 levels for a dosage series of CPDS in the presence of LPS.

### DETAILED DESCRIPTION

The invention provides treatment of inflammatory disease and disease states, using thione-forming disulfides (TFDs). A number of methods and compositions are discussed in the Summary of the Invention and further details are provided in this Detailed Description and in the Examples section. As would be readily appreciated by the skilled person, the disclosures can be read in combination.

Treatment of pathologies using thione-forming disulfides can include identification of a patient who would benefit from a reduction of inflammation, then selecting an appropriate TFD formulation, administering an appropriate TFD dosage, and monitoring the inflammation. Applicable patients with inflammation can be any experiencing classic symptoms of acute or chronic inflammation, including types of inflammation associated with immune responses overactive due to non-adaptive (innate) immunity phenomena. Appropriate formulations and methods of administration can depend on the site of the inflammation and status of the patient. The patient should be monitored to see the progress in inflammation reduction and possible toxicity (which is low, even at high dosages for many TFDs; see, e.g., Chemoterapia, Vol V, No. 4, 1986).

### Identification of Patients.

Patients with many forms of inflammation can benefit from treatment with TFDs. Inflammation can be due to, e.g., physical trama, an infection, an immune disorder, a hypersensitivity reaction, exposure to a chemical irritant, arthus reaction, graft versus host, exposure to radiation, and/or the like. That is, inflammation can occur in many different forms and from many causes. These causes can include but are not limited to pathogens (e.g., bacteria, viruses, parasites), physical insult or trauma (e.g., burns), exogenous foreign agents (e.g., asbestos, chemicals), endogenous foreign agents (e.g., immune complexes, autoimmune peptides or sequences, urate crystals). Patients with a viral pneumonia, particularly those patients with an overactive cytokine response, can benefit significantly from treatment with TDFs (such as CPDS). The inflammations can broadly be divided between inflammation due to a response of the adaptive immune system and responses of the innate immune system.

Identification of a patient in need of IL-1β, IL-1α, or IL-6 inhibition entails more than treating the patent in a way that reduces an IL-1 activity and/or IL-6 activity. Identification of a patient for IL-1 or IL-6 activity inhibition, e.g., using a TFD (such as CPDS) requires identification in a particular patient an indication, e.g., the caregiver (e.g., physician) determines is due to (or exacerbated by) an excessive IL-1 or IL-6 activity. With this information under consideration, the caregiver treats the patient with the TFD with the intention of lowering IL-1 and/or IL-6 activity in the patient, e.g., by at least 10%, 20%, 30%, 40%, 50%, 60%, 70%, 80%, 85%, 90%, 95%, or more.

Inflammatory responses of a patient can be divided into acute inflammatory response and chronic inflammatory response. Both types of response can generally be characterized by heat, pain, redness, and swelling. Severe forms of inflammatory responses can involve a network of positively reinforcing cytokines (e.g., IL-1, IL-6, IL-16 TNF, and/or IFN-γ, e.g., in a cytokine storm of inflammation.

Acute inflammatory responses can be generally characterized by vasodilation, increased vascular permeability, recruitment of neutrophils, and fever. Mediators of acute inflammatory responses, include but are not limited to complement (e.g., C3a, C3b, C4b, C5a, etc.), kinins, clotting and fibrinolytic proteins, lipid mediators (e.g., prostaglandins, leukotrienes, platelet activating factor (PAF), lipotoxins, peptides and amines (e.g., histamine, serotonin, neuropeptides, nitric oxide) and pro-inflammatory cytokines (e.g., IL-1, IL-4, IL-6, IL-8, IL-12, TNF-α, TNF-β, and IFN-γ). Other acute inflammatory responses can include inflammation caused by bacterial and/or viral infections, particularly pneumonia caused by members of the Coronavirus family, such as SARS-CoV-2 (COVID-19 agent). Acute inflammatory responses typically resolve within hours, days, or a few weeks. For example, inflammation from a mosquito bite may resolve in an hour, a sun burn in days, and a sprained joint in weeks.

Chronic inflammatory responses occur when acute inflammation has not been resolved over a period of time and can be generally characterized by an accumulation of macrophages and lymphocytes as well as growth of fibroblasts and vascular tissue. Several long term effects of chronic inflammation that can be observed are tissue scarring, formation of tissue granuloma, formation of tissue fibroma, and even initiation of inappropriate cell growth, such as in found in cancer. Chronic inflammation can result from re-injury, uncontrolled infection, autoimmune assaults, a malignancy, and/or the like.

In one embodiment, macrophages, neutrophils, and other phagocytic cells release cytokines in response to encounter with bacteria, which results in heat, pain, redness, and swelling. This response can be innate and not moderated by previous exposure of the patient to the bacteria pathogen. Exemplary innate (not adaptive immunity) inflammations can be present in sickle cell, trauma, diabetes, venom, initial infections by viruses or microbes, chemical burns, and the like. The cytokines can cause dilation and increased permeability of the blood vessels, which can result in increased blood flow and fluid leakage. Additionally, pro-inflammatory cytokines can cause increased expression of adhesion molecules on leukocytes and endothelial cells in blood vessel walls. The adhesion molecules include, but are not limited to, P-selectin, E-selectin, integrins, LFA-I, CR3, and P-CAM (CD31). The effect of this increased expression can cause leukocytes to become more adherent to blood vessel walls and allow migration of leukocytes into a local area containing the pathogen. Other factors that are released in response to a foreign pathogen, e.g., bacteria, include chemokines (e.g., RANTES, MIP, prostaglandins, peroxides, nitric oxide, and leukotrienes (which can include cyclooxygenase-2 and PAF).

Inflammation can be assessed by several methods known to one of skill in the art. Examples of methods that can be used to assess the presence of mediators of inflammation (e.g., cytokines such as IL-1 and IL-6, chemokines, lipid mediators, etc.) are enzyme-linked immunosorbent assay (ELISA), sandwich ELISA, cytometric bead array, and flow cytometry (which includes fluorescent activated, cell sorting or FACS). The presence of pro-inflammatory cytokines, chemokines, prostaglandins, peroxides, nitric oxide, leukotrienes, and PAFs can be detected by either method. ELISA kits for specific cytokines, chemokines, and other inflammatory factors, are commercially available (e.g., BD Pharmingen, San Diego, Calif.; Becton Dickinson, San Jose, Calif.). Likewise, antibodies specific for specific cytokines, chemokines, and other inflammatory factors are also commercially available (e.g., BD Pharmingen, San Diego, Calif.; Becton Dickinson, San Jose, Calif.; R&D Systems, Minneapolis Minn., and the like). Expression of adhesion molecules can also be detected using FACS. The expression of adhesion molecules can be monitored over time using biological samples taken from various locations, or alternatively from locations that are currently suspected of having an inflammatory response. Complement protein C5a is able to activate mast cells, which can cause them to release the granular contents contained within the mast cells. These granular contents can include histamine, serotonin, and leukotriene LTB4, which can also be detected by ELISA or FACS. In an alternative, inflammatory cells (e.g., neutrophils, macrophages or other phagocytes, lymphocytes, etc.) can be assessed or enumerated, e.g., by flow cytometry using biological samples taken from various locations which are inflamed or alternatively, from locations that are suspected of having an inflammatory response. A high concentration of neutrophils or other leukocytes in a local region can indicate an inflammatory response, particularly an innate response, particularly with normal lymphocyte values, particularly wherein the lymphocytes are not activated. Such techniques can be used to identify patients that can most benefit from TFD treatment, e.g., those with inflammation predominantly due to the activity of non-lymphocyte leukocytes, and particularly inflammation associated with macrophage activity and/or moderated by the cytokine IL-1 (α and/or β) or IL-6.

Identification of inflammation in a patient can also be assessed by physical examination. Several physical symptoms of inflammation include but are not limited to redness, pain, or swelling in a local region. These symptoms differ depending on what type of inflammation is occurring. In one aspect, inflammation can be associated with disease states. For example, inflammation can be associated with arthritis. Symptoms of arthritis include swelling of a joint, tenderness in inflamed joints, and pain associated with the swelling of joints. Other disease states in which inflammation has a role include, but are not limited to, psoriasis, diabetes, pneumonia, multiple sclerosis, sickle cell, breast hyperplasia, edema, cancers, and colonic polyps. Symptoms of psoriasis can include but are not limited to rash or redness on the skin surface, dry and/or irritated texture, itchiness, and discomfort. Symptoms of breast hyperplasia can include but are not limited to a buildup of a tissue mass in one or more breast. Symptoms of edema can include but are not limited to accumulation of fluid and some swelling of tissues at a site.

In another aspect, inflammation can occur in response to an agent to which an individual has been exposed. Examples of agents which can elicit an inflammatory response include but are not limited to poison oak, poison ivy, household cleaners, and chemical compounds. Exposure to such agents elicits symptoms which can include, but are not limited to, redness, itchiness, swelling, exudate formation, and development of a rash. Patients with these types of inflammation can typically benefit from TFD treatment and reduction of IL-1β activity.

Patients in need of reduction of IL-1β or IL-6 activity can be identified by outward physical observation, diagnosis, and/or clinical laboratory analyses, as described above. In a preferred embodiment, patients that will benefit from reduction of IL-1β activity with TFDs are broadly those with symptoms associated with inflammation other than solely immune memory (adaptive systems, such as those that establish humoral immunity and T-cell attacks) inflammation. Patients experiencing inflammation from trauma or inflammation initiated or moderated by innate immune system elements (e.g., activated non-lymphocyte cells, such as granulocytes, RES, microglia, and/or macrophages) are particularly suited to benefit from TFD treatments.

### Interleukin 1 (IL-1).

The interleukin 1 family includes IL-1α and IL-1β. Both IL-1 types are known to be pro-inflammation cytokines. IL-1 activity can influence a variety of different cell types. For example, endothelial cells can be stimulated to induce an inflammatory cascade by secreting chemokines such as MCP-1 and by increasing expression of vascular adhesion molecules such as E-Selectin, ICAM-1 and VCAM-1. MCP-1 can stimulate chemotaxis and activate mononuclear cell integrins, thus prompting mononuclear infiltration into an area of early inflammation. IL-1 can also induce expression of more IL-1 molecules in newly arriving monocytes, resulting in a positive feedback of increasing inflammation. IL-1 can enhance production of IFN-γ. IL-1 and IL-6 can positively feedback, e.g., one into the other, causing a spiral of inflammation. IL-1 can work with NK cells in conjunction with macrophage-derived IL-12 to induce IFN-γ activation of more macrophages. Further, IL-1 can also induce the expression of matrix metalloproteinases from local fibroblasts causing extracellular matrix degradation but also down-modulating the inflammation cascade by degrading IL-1β.

IL-1β is initially expressed as a 269 amino acid precursor that is processed into a 116 amino acid pro-segment and a 153 amino acid mature segment. The mature IL-113 segment is released to initiate a signal cascade generating local and/or systemic inflammation.

IL-1α is somewhat different from IL-1β in sequence and structure. IL-1α is initially synthesized as a 271 amino acid pro-cytokine that is enzymatically cleaved into the 159 amino acid active form and a 112 amino acid pro-sequence. It is notable that IL-1α can have bioactivity even in the intact pro-cytokine form, intracellularly and on the cell surface. On the cell surface, IL-1α can be a membrane-bound molecule, anchored and presenting from the cell membrane.

IL-1 is a “master” regulatory cytokine that is operative in both local and systemic inflammation (Dinarello Calif., Treating inflammation by blocking interleukin-1 in humans, Semin Immunol. 25(6):469-84). The availability of specific IL-1 targeting therapies that are active for an expanding list of diseases has provided insights into the pathologic role of IL-1-mediated inflammation. For example, when IL-1, (either IL-1α or IL-1β) was administered to patients, e.g., in order to improve bone marrow function or increase host immune responses against cancer, these patients experienced unacceptable high toxicity with fever, anorexia, arthralgias, myalgias, fatigue, gastrointestinal upset, sleep interruption, and hypotension. It is notable the inhibition of IL-1β can be useful in reversal the immunosuppression in mouse breast cancer and synergizes with anti-PD-1 for tumor abrogation. Blockade of IL-1 activity in multiple inflammatory syndromes can result in a rapid, sustained reduction in severity of several diseases. In conditions such as gout arthritis, IL-1 blockade can be effective therapy. Three IL-1 blockers have been approved: the IL-1 receptor antagonist, Anakinra, blocks the IL-1 receptor and therefore reduces the activity of IL-1α and IL-1β. A soluble “decoy receptor”, Rilonacept, and a neutralizing monoclonal anti-interleukin-1β antibody, Canakinumab, are also approved. A variety of additional monoclonal antibody targeting the IL-1 receptor and neutralizing anti-IL-1α have also been tested and/or are under development. However, the present IL-1 inhibition methods can provide more effective IL-1 inhibition at a lower cost with reduced toxicity. Further, complementary benefits in IL-1 inhibition, with lower toxicity can be provided with treatments combining CPDS with other inhibitors, such as those described above.

Due to excessive or inappropriate expression of IL-1, patients can suffer painful inflammation and ultimately degradation and damage of associated tissues. Thione-forming disulfides typically have low toxicity and can reduce IL-1 activity and associated inflammation, according to the present methods of treatment. A patient in need of TFD treatment can be identified according to the case history and present symptoms, and/or by clinical assay. For example, TFD treatment is appropriate for patients presenting with inflammation types known to be associated with innate immunity responses, particularly excessive inflammation caused by activated macrophages. Optionally, a patient in need of TFD treatment can be identified by clinical assays (discussed above) enumerating macrophages and/or IL-1 (α or β) levels in the patient.

### Interleukin 6 (IL-6).

The interleukin 6 (IL-6) is a pro-inflammatory cytokine that can take part in or initiate cytokine cascades in many inflammatory and auto-immune diseases. Because of the IL-6 role in pathologies, there has been interest in developing anti-IL-6 therapies. For example, tocilizumab is a monoclonal antibody against IL-6, possibly effective in reducing inflammation in a variety of disease states, such as rheumatoid arthritis.

More recently, IL-6 has been suspected of having a role in severe inflammatory episodes of COVID-19 infections and other viral respiratory infections (e.g., influenza, MERS, RSV, Rhinovirus, SARS, and the like. IL-6 appears to be a key cytokine triggering activation of other cytokines in a cascade referred to as a cytokine storm. Such episodes are associated with more serious morbidity and mortality outcomes.

We have found that thione-forming disulfides can reduce IL-6 activity at dosage levels well below non-toxic oral and intravenous dosing levels. A Coronavirus patient in need of TFD treatment can be identified, e.g., as having pneumonia and experiencing blood oxygen levels below about 90%. Optionally, TFD treatment can be appropriate when a patient has signs of a cytokine storm, e.g., elevated cytokines being produced by cells in response to a respiratory infection. In addition, a patient in need of TFD treatment can be identified by clinical assays (e.g., ELISA) detecting excessive IL-6 levels in the patient.

### Thione-Forming Disulfides

Thione-forming disulfides are used in the methods of the invention to reduce IL-1 activity and inflammation in a patient. Thione-forming disulfides are disulfides that upon reaction, for example with a thiol, give rise to a thione. The thione-forming disulfides generally react with thiols or sulfhydryl groups in an essentially irreversible reaction to give as products, a disulfide and a thione. The thione-forming disulfide may initially form a thiol, which tautomerizes to thione. The thione preferably does not equilibrate with the disulfide product. This reaction is exemplified below for the reaction of 6,6′-dithiodinicotinic acid with two thiol groups (R′SH), to produce two thiones and a disulfide product:

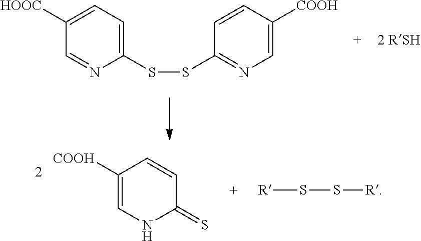

The thione-forming disulfides are, for example, dithiobis-heterocyclic compounds, optionally comprising aromatic heterocycles. The heterocyclic moiety can include, for example, 1 to 5 nitrogen atoms and optionally a further heteroatom, such as sulfur or oxygen, in the ring. The compound may comprise, for example, a cyclic group having at least one five- or six-membered heterocyclic ring, each heterocyclic ring comprising nitrogen, and optionally further heteroatoms such as N, O, or S. The dithiobis-heterocyclic compound may comprise, for example, a pyridinyl, pyrimidinyl or thiazolyl heterocyclic group. The heterocyclic group can be substituted or unsubstituted.

The heterocyclic ring can comprise or present, for example, negative or potentially negatively charged substituents, such as carboxyls, carboxylic esters, amides, sulfate, sulfonate or phosphate groups, or salts of any of the foregoing, or nitrile groups.

The thione-forming disulfides in one embodiment may be represented by the general structure: R—S—S—R, where R is organic moiety, and wherein R can be the same or different organic moiety. Thus, optionally the compound is a mixed disulfide. In one embodiment, the disulfide compounds are dithiobis-heterocyclic compounds R—S—S—R, wherein R comprises a heterocyclic aromatic group. In one embodiment, R comprises a cyclic group having at least one five- or six-member heterocyclic ring, each heterocyclic ring comprises one nitrogen and optionally additional heteroatoms, such as N, O, or S. R also can comprise single or fused rings, such as pyridine, pyrimidine, thiazole, oxazole, dithiouracil, 6-thioguanine, 6-mercaptopurine, and/or picoline.

In further embodiments, R comprises heterocyclic rings which may include negative or potentially negative substituents, such as carboxyl, carboxylic esters, amides, sulfate, sulfonate or phosphate groups, or salts of any of the foregoing, such as sodium salts, as well as nitro groups. In another embodiment, R is an unsubstituted or substituted pyridyl group, optionally substituted by anionic groups, alkyl groups, hydroxyl, —CN, halogen, CNO, carboxyl, ester, or amide groups.

In one embodiment, the thione-forming disulfides contain one of the following partial structures, a vinyl (C═C—N) type or a vinylidene (—C═C—C═N—) type structural fragment, and is proximally linked to a disulfide moiety, as represented in Scheme I, wherein X and Y represent atoms necessary to form a five- or six-membered substituted or unsubstituted heterocyclic ring and optionally fused to a substituted or unsubstituted carbocyclic or heterocyclic ring, permitting the formation of a thione, as indicated below in Scheme I:

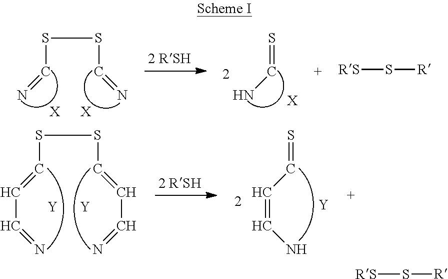

An example of a thione-forming disulfide is 6,6′-dithiodinicotinic acid (or carboxypyridine disulfide, herein designated as “CPDS”). In one embodiment, R comprises various substituted pyridyl or pyrimidyl moieties, with substituents including a carboxy group or salt form thereof, and/or ester and amide derivatives of the carboxy group.

Salt forms of any of the compounds may be used. Suitable salts are, for example, those that form with alkali or alkaline earth metals, with ammonia or with amines such as cyclohexylamine, morpholine or other aliphatic, alicyclic, aromatic or heterocyclic amines Salts include sodium, potassium, magnesium, and ammonium salts. As salt forms are typically ionized and dissociated in aqueous solutions (e.g., on administration to a patient), the selection among various salt alternatives is typically not expected to substantially influence the pharmacological activity of the associated TFD.

Suitable substituents which may be present on heterocycles of the disulfide compounds include ester moieties including methyl, ethyl and higher alkyl groups, cyclohexyl and other alicyclic groups. Suitable amide moieties include CONR′R″, wherein, R and R″ are independently H, aliphatic, heterocyclic, alicyclic or aromatic groups and substituted derivatives thereof.

The pyridine ring in 6,6′-dithiodinicotinic acid is merely one of the many possible thione-forming disulfides, e.g., that function in the treatments of the invention. Other heterocyclic aromatic moieties include quinolinyl, pyrimidinyl, and thiazolyl groups and substituted derivatives thereof, including various salt forms such as sodium, potassium, ammonium or magnesium salts. Non-limiting examples of thione-forming disulfides, including salts thereof, such as sodium salts, are listed in Table 1.

TABLE 1
EXAMPLES OF TFDs

PYRIDINE DERIVATIVES
6,6′-dithiodinicotinic acid
6,6′-dithiodinicotinic acid ester
2,2′-dithiobis-isonicotinic acid
2,2′-dithiobis-(5-acetamidopyridine)
2,2′-dithiobis
6,6′-dithiodinicotinamide
PYRIMIDINE DERIVATIVE
4-carboxypyrimidine-2-disulfide
THIAZOLE DERIVATIVE
Diethyl 2,2′-dithiobis-(4-thiazole carboxylate)

Thione forming disulfides described in the art may be used, for example, those described in U.S. Pat. Nos. 3,698,866; 3,597,160; 4,378,364; 4,152,439; 6,043,256; PCT WO 99/07368; Canadian Patent No. 985170; Grassetti, D. R., Cancer Letters, 31:187-195 (1986); Grassetti, D. R. Nature, 308(5959); Grassetti, D. R. Nature, 228(268):282-283 (1970), and U.S. Pat. No. 8,163,776, the disclosures of which are incorporated herein by reference in their entirety.

Exemplary useful thione-forming disulfides include the following compounds:

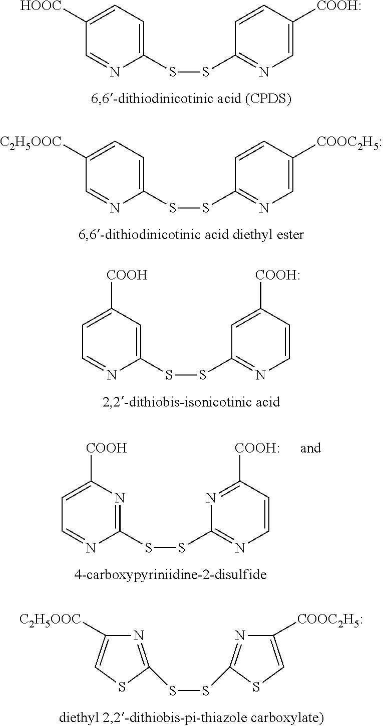

Other non-limiting examples include the compounds listed in Table 2, further including salts thereof, such as sodium salts:

TABLE 2

6,6′-dithiodipicolinic acid
4,4′-dithiodipicolinic acid
4,4′-dithiodinicotinic acid
2,2′-dithiodinicotinic acid
4,4′-dithiobis(2,5-pridinedicarboxylic acid)
4,4′-dithiobis(2,6-pyridinedicarboxylic acid)
2,2′-dithiobis-(3,4-pyridinedicarboxylic acid)
4,4′-dithiobis-(3,5-pyridinedicarboxylic acid)
4,4′-dithiobis-(2,3,5-pyridinetricarboxylic acid)
2,2′-dithiobis-(3,4,5-pyridinetricarboxylic acid
2,2′-dithiobis-(3,4,5,6-pyridinetetracarboxylic acid)

The synthesis of these disulfides can be often carried out by oxidation of the corresponding thiol with hydrogen peroxide or iodine-potassium iodide under neutral conditions. The thione forming disulfide compounds are available commercially or synthesized from commercially available compounds. Examples of methods for the syntheses of disulfide compounds, and commercial sources are as follows: 6-mercaptonicotinic acid (6MNA) is prepared according to Rath, C., Justus Liebig's Ann. Chem., 487, 95-106, (1931), and also is commercially available from Sigma-Aldrich, St. Louis, Mo. 6,6′-dithiodinicotinic acid (CPDS) is prepared as described by Grassetti et al., J. Med. Chem. 10: 1170-1172 (1967), and also is available commercially from different sources, such as Sigma-Aldrich (St. Louis, Mo.); Galantis S. P:A., Via delle Industrie, 11,30020, Marcon Venezia Italy; and Chemsyn Laboratories, 13605 W. 96th Terrace, Lenexa, Kans. 66215-1297.

The diethyl ester of 6,6′-dithiodinicotinic acid may be prepared as described by Grassetti, D. R., Cancer Lett., 31: 187-195 (1986). 4-Carboxypyrimidine 2-disulfide may be obtained by the oxidation of 2-mercapto-4-pyrimidine carboxylic acid, which may be prepared according to Daves et al., J. Herocyclic Chem., 23: 130133 (1964). The oxidation may be carried out according to the method of Fox and Gibas, J. Org. Chem., 23, 64-66 (1958). Diethyl 2,2′-dithiobis-4-thiazole carboxylate may be obtained by oxidation of ethyl 2-mercapto-4-thiazole carboxylate, and may be prepared according to D'Amico and Bartram, J. Org. Chem., 25, 1336-1342(1960). Isonicotinic acid is available commercially from Aldrich-Sigma (St. Louis, Mo.). See also, for example, Grassetti, D. R. et al. Journal of Medicinal Chemistry 9: 149 (1966); Grassetti, D. R. et al. Journal of Medicinal Chemistry 10:1170 (1967); and Grassetti, D. R. et al. Journal of Medicinal Chemistry 13: 273 (1970).

While not being limited to any theory, possible mechanisms of action of the thione-forming disulfide compounds in reducing inflammation generally include reaction of sulfhydryl groups on cell surfaces; modifying cellular surface interactions; and effects on the activity of the chromatin-bound enzyme, poly (ADP-Iibose) synthetase. See, e.g., Purnell, M. R. and Whish, W, J. D. Biochem. J., 85: 775-77.7 (1980); and Grassetti, D. R., Cancer Letters, 187-195 (1986). An interesting and unexpected mode of action particular to reduction of inflammation, e.g., in the context of patients experiencing excessive inflammation associated with innate immune systems, macrophages, IL-6, and/or excessive IL-1α or β as discussed herein, is the inhibition of IL-1, e.g., and/or the associated induced inflammation cascade.

Some thione forming disulfide compounds, such as CPDS, have been shown to have low toxicity. Grassetti, D. R., Cancer Letters, 31: 187 (1986); Boot, JH., Cell structure and Function, 20: 233 (1995); Grassetti, D. R., Drugs of the Future, 11: 559 (1986); U.S. Pat. No. 4,378,364; Grasserti D. R. and Murray, J. F., Biochem Pharmacal. 17(11): 2281-90 (1968); and Grassetti D. R. and Murray, J. F., Biochem Pharmacol. 16(12): 2387-93 (1967). For example, studies have shown IV doses up to 300 mg/kg (1 mM) are not fatal with only slight increase in kidney or liver weight; 2000 mg/kg oral doses did not result in substantial toxicity in mice. In one preferred embodiment, thione forming disulfides with low toxicity in individuals are selected for use in treatment regimens and for use in pharmaceutical compositions.

### Using Thione-Forming Disulfides for Treating Inflammation.

Inflammation can be treated by administering thione-forming disulfides (TFDs) to an individual with one or more types of inflammation. In one embodiment, the TFD is CPDS. In another embodiment, the TFD is 6,6′-dithiodinicotinic acid diethyl ester. In another embodiment, the TFD is 2,2′-dithiobis-isonicotinic acid. In another embodiment, the TFD is 4-carboxypyrimidine-2-disulfide. In yet another embodiment, the TFD is diethyl 2,2′-dithiobis-(4-thiazole carboxylate). The methods described above can be used to assess whether resolution of inflammation, either acute or chronic, has been achieved by administration of TFDs. For example, levels of pro-inflammatory cytokines can be monitored before and after the administration of TFDs and can be combined with physical examination of redness, swelling, etc., to ascertain if resolution of one or more inflammatory-responses has been achieved.

Various formulations of thione-forming disulfides (TFDs) may be used for administration. In one embodiment, the thione-forming disulfide is CPDS. In another embodiment, the CPDS is administered orally. The oral administration can be a capsule form or by dissolving CPDS powder in an aqueous solution for consumption (e.g., water). To make a capsule form, an effective amount of TFD, such as CPDS, can be admixed with solid or viscous ingredients disclosed surpa. In certain embodiments, the CPDS for oral administration is at least 50%, 75%, 90%, or more pure. In a preferred embodiment, 99.7% pure CPDS is used in capsule form for oral ingestion. In a preferred embodiment, 99.7% pure CPDS is in capsule form for oral ingestion. In another embodiment, the administration of TFDs help resolve acute inflammatory response by modulating the expression of inflammatory mediators such as IL-1α, IL-1β, IL-4, IL-6, TGF-β, IL-16, TNF, and IL-13.

These compositions may be formulated for administration by injection, e.g., intraperitoneally or intravenously. CPDS can precipitate when administered subcutaneously or intramuscularly, so administration intraperitoneally or intravenously is preferable. Accordingly, these compositions are for example combined with pharmaceutically acceptable vehicles disclosed supra. For example, the TFDs can constitute about 0.01% to 50% by weight of the formulation depending upon practical or empirical considerations such as solubility and osmolality. The particular dosage regimen, (e.g., dose, timing, and repetition), will depend on the particular individual and that individual's medical history. Dosage examples can include, but is not limited to a dose of less than about 10 ug to more than about 5 g TFD/kg body weight, or about 500 ug to about 1 g/kg body weight, or about 1 mg to about 900 mg/kg body weight, or about 50 mg to about 750 mg TFD/kg body weight, or about 100 mg to about 500 mg TFD/kg body weight. Empirical considerations, such as the half-life, generally will contribute to determination of the dosage. Other dosages, such as about 10 mg to 250 mg, e.g., 140 mg daily, are possible over a daily, weekly, monthly, or yearly dosing regimen. Doses up to 300 mg/kg (1 mM) intravenous and 2000 mg/kg oral are tolerated.

The purity of thione-forming sulfides can be an important factor for determining toxicity to an individual taking TFDs. For example, the purity less than or at least about 75%, or at least about 80%, or at least about 85%, or at least about 90%, or at least about 95%, or at least about 99%. For example, the purity may be 98.2%, 98.4%, 98.6%, or 98.8%. Some TFDs are sensitive to light, and therefore it is preferred that these TFDs be protected from light, for example, during storage.

The frequency of TFD administration may be determined and adjusted over the course of therapy, and can be based on presence of inflammatory cytokines (e.g., IL-1α, IL-10, IL-4, IL-6, IL-8, IL-12, IL-16, TNF-α, TNF-β, and IFN-γ), inflammatory cell counts (e.g., neutrophil, total macrophages, activated macrophages, etc.), or other immune cell counts (e.g., NK cells, T cells, activated beta lymphocytes, etc.), alleviation of symptoms (e.g., lessening in swelling, redness, fever, pain, etc.), or physical health, or psychological health (e.g., maintaining a sense of well-being). Cytokines and immune cell counts may be monitored by obtaining biological samples and assessing for the presence thereof by using western blot, bioassay, ELISA and FACS analysis or any other technique discussed above or known to a skilled artisan.

Other appropriate dosing schedules may be as frequent as multiple doses daily or 3 doses per week, or one dose per week, or one dose every two to four weeks, or one dose on a monthly or less frequent schedule depending on the individual. The dosing regimen can be sustained for a week, two weeks, a month, three months, six months, one year, or, for example, ten years. Sustained continuous release formulations of the compositions may be appropriate. Various formulations and devices for achieving sustained release are known in the art.

The TFD can be used alone to treat patients, or can be used in combination with other analgesics, IL-6 inhibitors, or IL-1 inhibitors. For example, the TFDs can be used in combination with previously known IL-1 inhibitors or IL-6 inhibitors. The combinations can have additive, complementary, or synergistic combined inhibitory effect. For example, the TFDs (such as CPDS) can be used in combination with therapeutics that target and IL-1 or IL-1 receptor. Benefits can be obtained by combination of CPDS with, e.g., antibodies against IL-1 receptors, antibodies against IL-1, canakinumab, anakinra, and rilonacept. TFDs can also inhibit IL-6, and can be used in combination with other IL-6 inhibitors, such as Tocilizumab.

The patient sample for determination of cytokines or cell count/morphology obtained at a local site of the inflammation and/or can be a generalized sample, e.g., of a body fluid. For example, the sample can be a swab, biopsy, or aspiration. The body fluid sample can be an exudate, drainage, cerebrospinal fluid, whole blood, plasma, synovial fluid, urine, sputum, and/or the like. Where determination of IL-1 levels is of interest, the levels can be determined or estimated by an absolute count of macrophages, a count of activated macrophages, a ratio of macrophages to lymphocytes, or a ratio of activated lymphocytes to activated macrophages. Alternately, a level of IL-1 (α and/or β) or IL-6 can be determined directly in a sample by quantitating an IL-1 molecular concentration (e.g., GC/MS, ELISA, Luminex, or antibody labeling), or by measuring an IL-1 or IL-6 activity level (e.g., bioassay).

TFDs and CPDS are typically administered in an amount which is clinically effective yet not toxic to the individual. Toxicity may be determined by a stepwise increment of dosage and monitoring survival rates, if an animal model is used, or in individuals, self-described state of physical health, or monitoring blood contents for indices of toxic response.

### Using Thione-Forming Disulfides for Treating Cancer.

It is notable that IL-1 can influence the appearance and propagation of certain cancers. That is, the ability of TFDs to inhibit IL-1 activities can be used to reduce cytokine cascades and irritant inflammations known to perpetuate cancers.

Over the years there has been debate as to whether IL-1β either inhibits or contributes to malignancies (reviewed in: Bent R; Interleukin-1 Beta-A Friend or Foe in Malignancies?, Int J Mol Sci. 2018 Jul. 24; 19(8)). This is related to the fact that IL-1β is triggered in response to inflammatory signals in a variety of immune cell types. Only IL-1β and IL-18 are cytokines processed by caspase-1 after inflammasome-mediated activation. IL-1β expression and its multiple roles in pathophysiological conditions can be interpreted in various ways. For example, IL-1 signaling activates innate immune cells including antigen presenting cells and drives divergence of CD4+ T cells towards T helper (Th) types Th1 and Th17 cells, and has been reported to be beneficial in resolving acute inflammations, and to initiate adaptive anti-tumor responses. In contrast, some investigations have suggested that IL-1β produced during chronic inflammation supports tumor development (as can chronic inflammations regardless of source). Importantly, IL-1β that is produced within the tumor microenvironment (largely ascribed to tumor-infiltrating macrophages) has been implicated in promoting tumor growth and metastasis via several candidate mechanisms including by expression of IL-1 targets which promote angiogenesis and induction of soluble mediators in cancer-associated fibroblasts that evoke antiapoptotic signaling in tumor cells. Others have opined that IL-1β promotes the propagation of myeloid-derived suppressor cells. Other investigators have noted that elevated expression of IL-1β induces invasiveness of human triple negative breast cancer cells and can be suppressed by a plant-derived inhibitor of breast cancer cell that has been suggested to inhibit IL-1β inhibition (Jeon, H., Han, J. Nam, S. J., Lee J. E. and Kim, S.: Elevated IL-1β expression induces invasiveness of triple negative breast cancer cells and is suppressed by zerumbone, Chem Biol Interact. 2016 Oct. 25; 258:126-33). It is not unreasonable to conclude that IL-1 inhibition, at least in the context of chronic inflammation can be used to reduce the incidence of certain cancers, and IL-1 inhibition can be used to inhibit growth of malignant tumors.

For example, it is notable that IL-1β inhibition has been found useful in certain cancers, such as human triple negative breast cancers. IL-1β inhibition can reverse the immunosuppression in mouse breast cancer and synergizes with anti-PD-1 for tumor abrogation. See, e.g., PNAS 116 (4): 1361-1369; 2019. IL-1β is abundant in the tumor microenvironment of 4T1 breast cancer cells, promoting tumor growth, but also antitumor activities.

CPDS is an active agent with the capacity for pleiotropic modulation of various biochemical pathways as well as modulation and impact on various components of the immune response, including for example poly (ADP-ribose) polymerase (PARP) inhibition as well as activation of various cells of the immune response, e.g., activation of NK cells. Data described herein find that that CPDS acts to block IL-1β function, suggesting a new method of preventing or reducing tumor growth by applying TFDs, such as CPDS) when it is suspected IL-1β is making a significant contribution to a cancer pathology in a patient. We hypothesize that the ability of CPDS to down regulate IL-1β underscores an important component of its anti-cancer therapeutic potential, including for human breast cancer.

A recent large, multi-center and comprehensive clinical trial examined the effect of IL-1β inhibition using Canakinumab on pulmonary cancer in patients with atherosclerosis in a randomized double-blind, placebo-controlled trial (Ridker, P M et al., (Lancet. 2017 Oct. 21; 390(10105):1833-1842). The results built on earlier hypotheses in the literature that inflammation in the tumor microenvironment mediated by IL-1β could have a role in cancer invasiveness, progression, and metastases. This study focused on an additional analysis in the Canakinumab Anti-inflammatory Thrombosis Outcomes Study (CANTOS), a randomized trial of the role of interleukin-1β inhibition in atherosclerosis, with the aim of establishing whether inhibition of a major product of the Nod-like receptor protein 3 (NLRP3) inflammasome with canakinumab might alter cancer incidence.

As our data herein shows, CPDS blocks production of IL-1β. CPDS is therefore identified as an agent expected to have substantial influence over the appearance, and progression of many cancers (e.g., hematological malignancies and macrophage-infiltrated tumors, such as many lung cancers). The suitability of CPDS is underscored by the observations noted elsewhere in the patent application is that a concentration as low as 50 uM CPDS blocks the secretion of 94% of IL-1β.

### Pharmaceutical Compositions.

The disulfide compounds, including salts thereof, are optionally provided in a pharmaceutically acceptable form with a pharmaceutically acceptable carrier, for example in a pharmaceutical dosage form, pharmaceutical grade organic or inorganic carriers can be used. Preferred pharmaceutically acceptable salts are salts which retain the activity of the compound and do not impart any deleterious or untoward effect on the subject to which it is administered and by the context in which it is administered. Often, a preferred carrier is buffered at a basic pH.

Preferred pharmaceutically acceptable carriers are those which do not cause an intolerable side effect, but which allow the thione-forming disulfide compound to stably retain pharmacological activity in storage and/or in the body. Formulations for parenteral and non-parenteral drug delivery are known in the art and are set forth in, e.g., Remington: The Science and Practice of Pharmacy 20th Ed. Mack Publishing (2000).

For solid compositions, conventional non-toxic carriers include, for example mannitol, lactose, starch, magnesium stearate, magnesium carbonate, sodium saccharin, talcum, cellulose, glucose, sucrose, pectin, dextrin, tragacanth, methyl cellulose, sodium carboxymethyl cellulose, a low-melting wax, cocoa butter, and the like may be used. The active compound, as defined above, may be formulated as suppositories using, for example, polyalkylene glycols, for example, propylene glycol as a carrier.

A solid carrier can be one or more substances which may also act as diluents, flavoring agents, solubilizers, lubricants, suspending agents, binders, or tablet disintegrating agents; it can also be an encapsulating material. In a similar manner, cachets or transdermal systems are included. In powders, the carrier is a finely divided solid which is in a mixture with the finely divided active component. In tablets, the active component can be mixed with the carrier having the necessary binding properties in suitable proportions, for pressing into the desired shape and size.

Liquid form preparations include solutions, suspensions, or emulsions suitable, e.g., for oral administration. Aqueous solutions for oral administration can be prepared by dissolving the active compound in water and adding suitable flavorants, coloring agents, stabilizers, and thickening agents, as desired. Aqueous suspensions or emulsions for oral use can be made by dispersing the finely divided active component in water together with a viscous material such as natural or synthetic gums, resins, methyl cellulose, sodium carboxymethyl cellulose, and other suspending agents known to the pharmaceutical formulation art. If desired, the pharmaceutical composition to be administered may also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art, based on the teachings herein. For example, see Remington's Pharmaceutical Sciences supra. The composition or formulation to be administered will preferably contain a quantity of the active compound in an amount effective to alleviate the symptoms of the subject being treated.

Parenteral administration is generally characterized by injection, e.g., whether subcutaneously, intramuscularly, or intravenously. Injectables can be prepared in conventional forms, either as liquid solutions or suspension, solid forms suitable for solution or suspension in liquid prior to injection, or as emulsions. Suitable excipients include, for example, water, saline, aqueous dextrose, glycerol, ethanol, or the like. In addition, if desired, the pharmaceutical compositions may also contain minor amounts of non-toxic substances such as wetting or emulsifying agents, auxiliary pH buffering agents and the like, for example, sodium acetate, sorbitan monolaurate, triethanolamine oleate, and/or the like.

The pharmaceutical preparation may be in unit dosage form. In such a form, the preparation is divided into unit doses containing appropriate quantities of the active component. The unit dosage form can be a packaged preparation containing discrete quantities of the preparation, for example, packaged tablets, capsules, and powders in vials or ampoules. The unit dosage form can also be a capsule, cachet, or tablet itself, or it can be the appropriate number of these packaged forms.

Other formulations include those suitable for topical or transdermal administration, which may be suitable if the thione-forming disulfides are able to cross the mucosa. Topical administration can be achieved by combining the TFD, for example, CPDS, with one or more ingredients well known in the art that are suitable for topical or transdermal administration. Possible ingredients include, but are not limited to, petroleum jelly, glycerin, and any commercially available lip ointment. The amount of TFD, such as CPDS, can vary from treatment to treatment, however, an effective amount of CPDS is combined with ingredients suitable for topical or transdermal administration, as shown in the Examples section. Other formulations include suitable delivery forms known in the art including, but not limited to, aerosol formulations and carriers such as liposomes. See, for example, Mahato et. al. (1997), Pharm. Res. 14:853-859. Liposomal preparations include, but are not limited to, cytofectins, multilamellar vesicles, and unilamellar vesicles; and can include both PEGylated as well as non-PEGylated varieties.

The optimal effective concentration of thione-forming disulfides can be determined empirically and will depend on the type and severity of the disease, route of administration, disease progression and weight and mass or body area of the individual. Such determinations are within the skill of one in the art.

### Animal Models.

Use of animal models facilitates identification of active drug candidates, target levels of dosing, and candidates expected to have reduced toxicity. For example, most antibiotics, hormones, cytokines, and neurotransmitters that have an activity in a human also have an activity in a mouse. Although pharmacokinetics may differ in the animal models, animal studies are useful in screening activity, toxicity, benefits, and dosages. Follow up, e.g., phase 1 clinical studies, can be guided by prior animal studies, allowing fine tuning of administration parameters in human patients.

In certain cases, animal models may be used to mimic the type of inflammation that occurs in humans for purposes of testing effects or dosages of TFDs. A mouse model of arachidonic acid-induced inflammation may be used as exemplified in Example 6. Other mouse models may be used, for example, carrageenen-induced edema disclosed in Example 7. For each type of animal model of inflammation, TFDs (e.g., CPDS) can be administered in the experimental group (control group would receive no TFDs) and biological samples and observation would be assessed for efficacy of TFDs on the inflammation. Using animal models can also be useful for assessing toxicity to particular organs or systems.

### EXAMPLES

The following examples are offered to illustrate, but not to limit the claimed invention.

### Example 1—General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds. 5th edition, 1996); Gene Transfer Vectors for Mammalian Cells (J. M. Miller & M. P. Calos, eds., 1987); Current Protocols in Molecular Biology (P. M. Ausubel et al., eds., 1987); PCR: The Polymerase Chain Reaction, (Mullis et al., eds., 1994); Current Protocols in Immunology (ie. Coligan et al., eds., 1991); The Immunoassay Handbook (David Wild, ed., Stockton Press NY, 1994); Antibodies: A Laboratory, Manual (Harlow et al., eds., 1987); Methods of Immunological Analysis (R. Masseyeff, W. H. Albert, and N. K. Staines, eds., Weinheim: VCR Verlags gesellschaft. mbH, 1993), Immunobiology (Janeway, C. A. and Travers, P., 1997); and Fundamental Immunology (Paul, W. E., ed., 1999); Advanced Organic Chemistry, John Wiley & Sons, New York, 1985; and House, Modern Synthetic Reactions, The Benjamin/Cummings Publishing Company, Menlo Park, Calif., 1972.

### Example 2—Use of CPDS for Inhibition of IL-1β Activity

This Example summarizes laboratory test results of the thione-forming disulfide, 6,6′-dithiodinicotinic acid (CPDS), with respect to secretion and receptor blocking of the cytokine Interleukin 1 beta (IL-1β). Testing was conducted by SBH Sciences in Natick Mass., under contract to the Grassetti Family Trust, in January and February, 2019.

Methodology: The study included replicated tests of IL-1β levels with and without CPDS, as well as reference testing of untreated cells and cells treated with lactoferrin and clobetasol. A third test was conducted of IL-1 receptor agonist (IL-1RA) receptor, to assist in distinguishing whether reductions in IL-1β levels were associated with secretion or receptor blocking (via the receptor antagonist). Cell viability studies were conducted for both sets of tests.

Each of the tests used an ELISA model with the following protocols.

1. THP-1 cells were grown in (RPMI+10% FBS, 10 mM HEPES, 1 mM Sodium Pyruvate, and 0.05 mM 2-Mecaptpethanol) to P8.
2. Cells were plated at 30,000 cells per well in 120 ul with Media+20 ng/ml PMA+2× Genta.
3. Cells were incubated for 3 days at 37° C. and 5% CO2.
4. Cells were treated according to plan below.
5. Cells were incubated for three more days at 37° C. and 5% CO2.
6. Cell supernatant were collected and ELISA was measured for IL-1RA, IL-1α, and IL-1β.
7. Plate was read at 490 nm until O.D reached 1.5.

Day 1—1. THP-1+20 ng/ml PMA 30,000 cells per well 120 uL

Day 3—1. Add Compound at 4× in 60 uL. Incubate for 2 hours. CPDS—800 uM (200 uM Final), lactoferrin—800 ug/mL (200 ug/ml Final) clobetasol −40 ng/mL (10 ng/mL Final).

2. Add 30 uL IFNγ at 8×—20 ng/mL (2.5 ng/ml final) in 30 uL (Wells 2-10)
3. Add 30 uL LPS at 8×—20 ng/ml (2.5 ng/ml final) in 30 uL (Wells 2-10)
4. Incubate for 3 days.

Day 6—1. Collect 80 ul of sample 2 times and freeze for ELISA.

2. Develop with 15 uL of Substrate and read at 490 nm until plate reaches 1.5 OD.

An ELISA test uses components of the immune system (such as IgG or IgM antibodies) and chemicals for the detection of immune responses in the body (for example, to infectious microbes). The ELISA test involves an enzyme (a protein that catalyzes a biochemical reaction). It also involves an antibody or antigen (immunologic molecules) that may form an antigen-antibody reaction to provide a positive result or, if they do not react, a negative result.

THP-1 is a spontaneously immortalized monocyte-like cell line, derived from the peripheral blood of a childhood case of acute monocytic leukemia, which has been extensively used to study monocyte/macrophage functions, mechanisms, signaling pathways, and nutrient and drug transport. This cell line has become a common model to estimate modulation of monocyte and macrophage activities.

Summary of Results. Both tests of IL-1β levels showed CPDS reducing measured levels 92-99% at concentrations of 12.5 to 200 μM. This represents a complete neutralization of IL-1β. Test results are summarized in Table 3, below:

TABLE 3
INHIBITION OF PRODUCTION OF IL-1β BY CPDS

| | CPDS | | | | | | | |
|---|---|---|---|---|---|---|---|---|
| | Concentration | | | | | | | |
| | Ave IL-Ib | 1/10 | 1/10 | 1/10 | 2/28 | 2/28 | 2/28 | Relative |
| Treatment | in ng/ml | Rep 1 | Rep 2 | Avg | Rep 1 | Rep 2 | Rep 3 | % |
| CPDS (uM) + | 200.0 | 0.01 | 0.01 | 0.01 | 0.02 | 0.02 | 0.02 | 6.8% |
| 10 ng/ml | 100.0 | 0.01 | 0.01 | 0.01 | 0.03 | 0.01 | 0.02 | 8.2% |
| IFNy + | 50.0 | 0.01 | 0.01 | 0.01 | 0.02 | 0.01 | 0.02 | 6.1% |
| 2.5 ng/ml LPS | 25.0 | 0.05 | 0.03 | 0.04 | 0.04 | 0.03 | 0.04 | 13.7% |
| | 12.5 | 0.09 | 0.06 | 0.08 | 0.13 | 0.11 | 0.12 | 45.8% |
| | 6.3 | 0.15 | 0.14 | 0.15 | 0.23 | 0.24 | 0.24 | 88.7% |
| | 3.1 | 0.21 | 0.17 | 0.19 | 0.30 | 0.26 | 0.28 | 105.2% |
| | 1.6 | 0.19 | 0.18 | 0.19 | 0.32 | 0.29 | 0.30 | 114.2% |

Cell viability in CPDS-treated cells in both tests was at or over 100% at all CPDS concentrations except 200 μM, where there was a slight decline in viability, as can be seen in Table 4 below (nearly identical results were obtained in a second test, not shown).

In order to determine whether the reduction in IL-1β was from blocking of secretion or from blocking of the receptor, a human IL-1RA (receptor antagonist) ELISA test was performed. This test found that at all concentrations lower than 100 μM, CPDS did not affect IL-1RA production. CPDS was found to reduce IL-1RA by 60-76% at levels of 100-200 μM, respectively.

Conclusions. CPDS was found to completely neutralize the production of IL-1β in human cells at concentrations ranging from 50-200 μM, with near neutralization at 25 μM. Because CPDS had no effect on the IL-1β receptor antagonist at levels below 100 μM, this reduction was not due to effects on the receptor. Therefore, the effect of CPDS on IL-1β must be on the cell's secretion of the cytokine. In summary, CPDS was found to fully neutralize the secretion of IL-1β.

### Example 3—Use of CPDS for Treatment of Breast Hyperplasia

A female individual, age 50, diagnosed with non-malignant intraductal breast hyperplasia on the upper left quadrant of the left breast was treated with CPDS. The individual had been experiencing pain, mostly in the left breast and left axilla for about 4-5 years prior to treatment with CPDS. CPDS was administered orally for forty-eight months, at a dose of 140 mg per day. CPDS was administered in 99.7% pure form in capsules without excipients.

Mammograms showed that the breast hyperplasia stopped growing after the individual took CPDS. No side effect upon taking CPDS at the amount administered was reported. Pain subsided within two days of commencing the CPDS regimen. The pain was eliminated while the individual was on a daily dose of CPDS. Following discontinuation of CPDS, the pain returned within two to three days.

### Example 4—Use of CPDS for Treatment of Arthritic Psoriasis

The patient was a 37-year old male. His disease began 1 year previously. His main complaint was pain in his right wrist, which prevented him from using his right hand and arm; he had bothersome itching cutaneous psoriatic lesions. Treatment at the time consisted of cortisone (5 mg/day) and non-steroidal anti-inflammatory drugs and weekly administrations of methotrexate (MTX) 5 mg/week. The side effects of MTX were severe stomach, throat, and mouth lesions, nightmares, visual hallucinations, and dizziness.

The patient began taking oral CPDS 90 days previous (140 mg/day), 4 days a week. Within a few days, the side effects of MTX became much milder, and his immune defenses increased. Instead of catching a cold, as he had often, that lasted for weeks and needed antibiotics, his colds were much more brief normal duration.

The patient has abandoned non-steroidal anti-inflammatory drugs, reduced his dosage of cortisone to 2 mg/day, and abandoned all topical medication on his psoriatic lesions. He feels healthy but still continues MTX and cortisone. He had regained function of his right hand and wrist.

### Example 5—Use of CPDS for Treatment of Colonic Polyps

A female individual, age 89, was diagnosed with colon neoplasia. About one month after the initial diagnosis, the individual underwent semi colotomy at which time about 45 cm of her colon was removed. About 3 months later, a check-up visit showed new extensive growth of polyps. The polyps were cauterized at that time.

About one month later, the individual began treatment with CPDS which was administered orally at a dose of 140 mg per day. The individual showed no new growth of polyps during check-up visits at 1, 3, 9, and 10 months after the commencement of CPDS treatment.

### Examples 6-10, the Following General Techniques and/or Protocols are Used

CPDS was administered in doses of 600 mg/kilo/day to animals prior to induction of the physiologic response of either inflammation or pain. This protocol is based on CPDS being administered orally. CPDS is finely ground and mixed with powdered chow in a ratio of 4 mg CPDS per gram of chow and then re-shaped. Mice consume about 3 grams of chow per day (equivalent to about 12 mg CPDS or 600 mg/kg). CPDS can also be administered by intraperitoneal injection of the same amounts of CPDS dissolved in buffer or sodium bicarbonate solution. The solution can be given every 4 hours or at longer intervals.

Female BALB/c mice were 6 to 8 weeks old when first acquired from Jackson Labs (Bar Harbor, Me.). Following two weeks of acclimatization, the mice are identified and randomized into 2 groups. The mice are fed ad libitum. At the end of each experiment, mice are sacrificed by CO₂ anoxia. The protocol can include collection of biological samples (e.g., blood and/or urine and/or tissue) for other analysis.

### Example 6—Use of CPDS Against Arachadonic Acid-Induced Inflammation

A mouse model of arachidonic acid-induced inflammatory response is used. Swelling of the ears of mouse is induced by arachidonic acid. Arachidonic acid is painted on the inner surface of the ear of the mouse. CPDS was administered orally at a dosage of 600 mg/kilo/day 24 hours prior to arachidonic acid treatment. Subsequent swelling of the ear was measured with a caliper at different times following the application of arachidonic acid.

### Example 7—Use of CPDS for Treatment of Carrageenan-Induced Edema

In a second model using mice, edema in the footpad is induced by carrageenan. The mice are injected with 0.1 ml, saline continuing 1.0% carrageenan in the left rear footpad. CPDS was administered orally 24 hours and 2 hours before injection at a dosage of 600 mg/kg. The thickness of each paw is measured by plethysmography at different times following the injection of carrageenan (60 minute intervals).

### Example 8—Use of CPDS for Treatment of Arthritis

In a third model, using Wistar rats, arthritis is induced by Freund's adjuvant. The thickness of the ankle and wrist are evaluated before the induction as a pre-immunization baseline value. The rats are injected intracutaneously with the adjuvant in the central dorsal area at the base of the tail. The rats are evaluated from day 10 to 25 following the induction. The intensity of arthritis is scored through the assessment of walking ability, skin redness and swelling of ankle and wrist joints. CPDS was administered orally stating 24 hours before injection and continued until end of experiment (4 mg/kilo in chow, ad libitum).

### Example 9—Analgesic Effects of CPDS

Analgesic effects are tested in in vitro efficacy studies using mice. An analgesic test using the hot plate model is used. Rats are placed in a glass cylinder on a heated plate (56±1° C.). The time between placement of each rat on the hot plate and subsequent licking of the paws or jumping is recorded as hot plate latency. A latency of 30 seconds is defined as complete analgesia.

### Example 10—Use of CPDS for Treatment of Inflammation in Macrophages

An in vitro model of inflammation mediated by macrophages is used. Primary macrophages isolated from humans or non-human species (e.g., mice or rats) were used. A murine macrophage cell line RAW264.7 (CCRL-2278) was also used. Macrophages are treated with CPDS. Then 90 minutes after treatment with CPDS (e.g., 50 μM), macrophages are stimulated with LPS (10 ug/ml) lipopolysaccharide for 24 to 48 hours to induce inflammatory responses. Then the effect of CPDS on inflammation is assessed by comparing the levels of anti-inflammatory mediators IL-6 and IL-10. IL-10 and IL-6 were determined by ELISA. The level of IL-6 and IL-10 were compared to the level prior to CPDS treatment. CPDS dramatically reduced the expression levels of IL-10 and IL-6.

We demonstrated that pre-treatment with CPDS prior to the addition of LPS (using murine RAW 264.7 cells) drastically reduced the secretion of IL-6. In addition we measured decrease in the secretion of IP-10. In this experiment, we have also measured cell viability and clearly demonstrated no cytotoxicity. Thus, this reduction in cytokine release in not due to cell inhibition/killing. Our positive control-Dexamethasone (DEX) was active as expected, and confirms the validity of the assay. In the case of IL-6, CPDS was more active vs. DEX.

### Example 11—Use of CPDS for Treatment of Poison Oak Exposure

A female individual, age 73, was exposed to poison oak on her left arm through physical contact. She developed a rash and other symptoms of inflammation such as redness, itching, burning sensation, and swelling. The individual was treated using topical application a cream containing 1% CPDS by weight. The cream was applied every two hours on the first day, and thereafter two to three times a day. The cream contained water, mineral oil, stearic acid, triethanolamine, propylene glycol, glyceryl stearate, stearyl alcohol, dirnethicone, isopropyl palmitate, cetyl alcohol, lactic acid, retinyl acetate, tocopheryl acetate, hydrolyzed collagen, fragrance, methylparaben, and O&C red #33. The individual was treated alternatively with 1% CPDS by weight in an ointment containing white petroleum and mineral oil.

The itching and burning dissipated immediately following topical application. The redness and swelling disappeared in about one week. The rash did not spread to other parts of the body. Symptoms did not recur at a later time.

### Example 12—Topical Treatment of Insect Bites

A female individual, age 65, was stung by a wasp on her hand. Topical application of a cream containing 2% CPDS by weight brought immediate relief; the burning and itching disappeared. Application of CPDS cream was repeated after two hours and every 4 hours thereafter. The swelling and redness vanished after 24 hours.

### Example 13—Treatment of Coronavirus Infections

Studies have shown that the Coronavirus family, including, e.g., SARS-CoV-2, SARS, and MERS, often present with a certain combination of cytokines in a “cytokine storm” of inflammatory messages. Such a storm of, e.g., interleukins, interferons, and chemokines in a coronavirus patient's lungs can cause a run-away auto-toxic inflammation destroying membranes and filling the lungs with fluids. Untreated cytokine storms can increase the likelihood of lung damage and/or death of the patient.

This run-away cytokine phenomenon can be so severe as to require throttling down of the immune response to the Coronavirus infection, e.g., with corticosteroids, such as prednisone. This, even in the face of potential additional spread of the virus and/or secondary infections.

In an aspect of the invention, a thione-forming disulfide (such as CPDS) is administered to a Coronavirus (e.g., SARS-CoV-2) patient to prevent or treat a cytokine storm event in the lungs of a patient. These benefits can be obtained, e.g., while maintaining substantial immune response against the virus itself, and against secondary invaders. The CPDS can be administered at levels to significantly reduce, directly or indirectly, the patient levels of IL-1, IL-4, IL-6, IL-8, IL-12, TNF-α, TNF-β, and/or IFN-γ. CPDS is expected to be a better treatment because it has fewer side effects than steroids, while providing substantial benefits in reduction of interleukins, necrosis factors, and the like.

Thus, CPDS treatment can be beneficial in any case of a cytokine storm. In particular, treatment of excess cytokines in a Coronavirus patient's lungs can include treatments with thione-forming disulfides orally, by injection, or by inhalation, in an amount effective in reducing symptoms of excess cytokines. In an embodiment, CPDS is administered in dosages to prevent elevated level of IL-1 or IL-6 activity in the lungs, or to reduce levels of IL-1 or IL-6 activity in the lungs to normal levels (e.g., those existing in the patient prior to the Coronavirus, e.g., Covid-19 infection).

### Example 14—Treatment of Cytokine Storm Involving IFN-γ

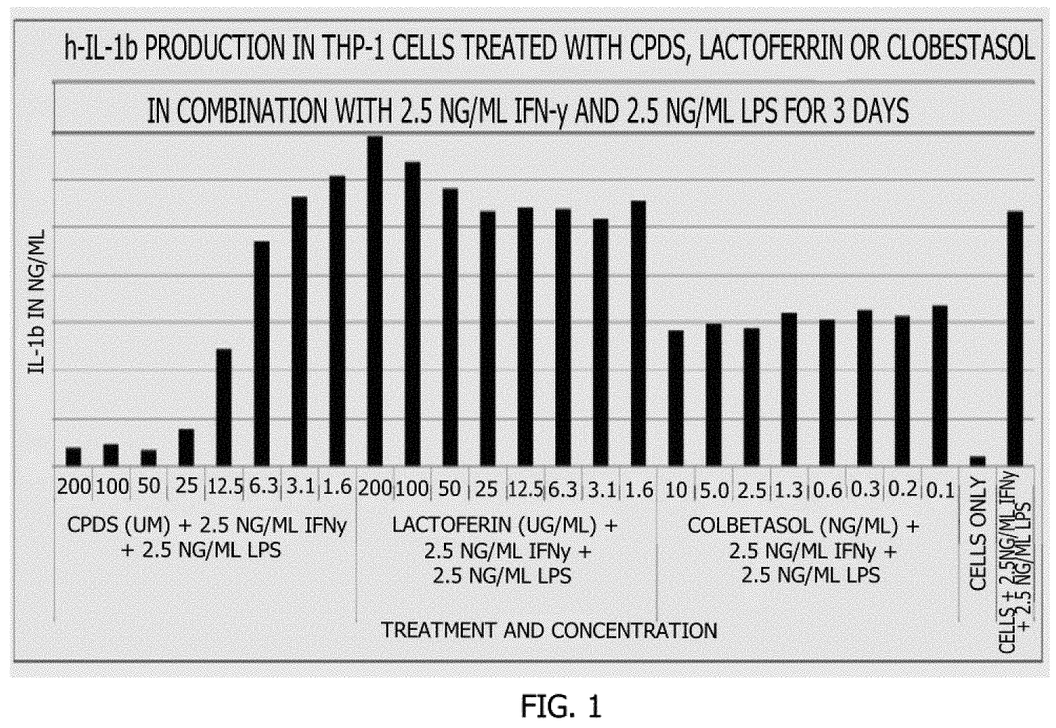

IFN-γ is a key cytokine involved in cytokine storms generated in the context of Coronavirus infections. We have tested the effect of CPDS on IFN-γ levels for cells inflamed by the presence of LPS (which substantially raises levels of IFN-γ). As shown in FIG. 1, clinically obtainable levels of CPDS markedly reduce the ability of IFN-γ to stimulate the production of IL-1β, associated with certain cytokine storm events.

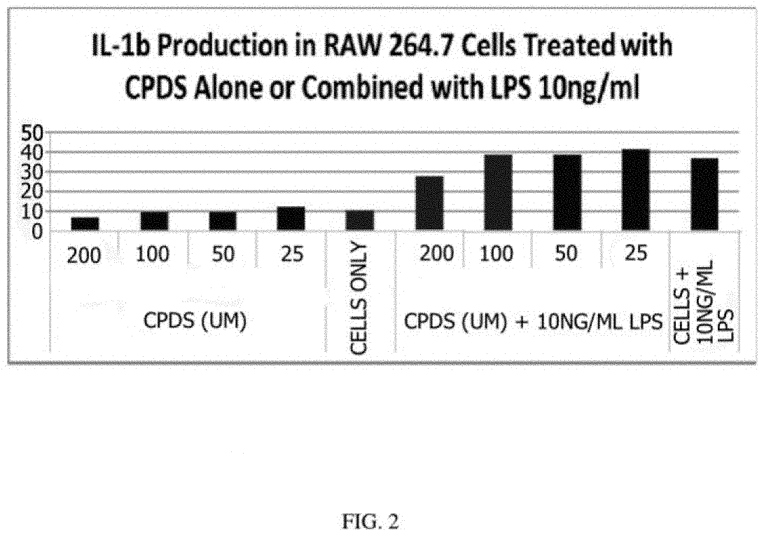

Further, it is notable that CPDS can directly reduce levels of IL-1β in normal cells and in inflamed cells. For example, as shown in FIG. 2, the presence of clinically effective amounts of CPDS can substantially reduce levels of IL-1β, e.g., particularly in situations of excessive inflammation, as can exist in the presence of bacterial lipopolysaccharides (LPS).

The ability of CPDS to reduce the influence of IFN-γ, LPS, and IL-1β can provide a lifesaving contribution to treatments against cytokine storm episodes.

Because of the effect shown in this experiment, we believe CPDS and related TFDs can provide substantially safe and effective treatments in certain cytokine storm episodes, e.g., such as those associated with Corona viruses, particularly SARS-CoV-2.

### Example 15—Treatment of Cytokine Storm Involving IL-6

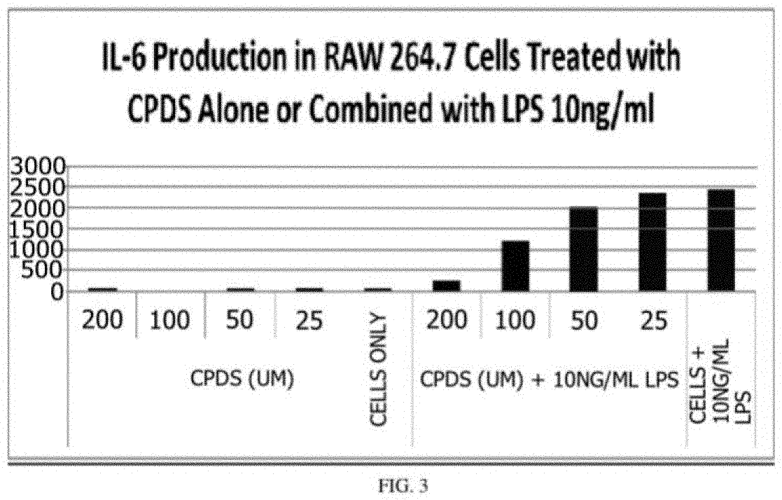

In our studies of TDF effects on cytokine production, we were surprised to find IL-6 levels are dramatically reduced in the presence of CPDS. See FIG. 3.

IL-6 is a key cytokine that can induce a wave of other cytokine activations in cytokine storms associated with certain infections. In particular, IL-6 is a key link in the sequential activation of an array of downstream cytokines in the cytokine storms associated with Coronavirus lung infections. This over-stimulation of cytokines can cause a layer of mucus, cells, and fluids to develop and block diffusion of oxygen through alveoli to the bloodstream.

CPDS, at clinically available concentrations, can substantially reduce the activation of IL-6. As shown in FIG. 3, macrophages inflamed by LPS produce abundant IL-6. However, in the presence of CPDS there is a concentration dependent diminution of IL-6 expression. As IL-6 is an important problematic cytokine in Coronavirus infections, we expect CPDS treatment of COVID-19 patients with CPDS can provide significant reductions in inflammation symptoms and reduce mortality.

### Examples 16—Embodiments

The novel contributions described herein include a variety of exemplary embodiments.

For example, the methods of treating a disease state can include identifying a patient with an IL-1β inflammation pathology clinical indication, and administering an effective dose of a thione-forming disulfide to the patient. Thereby IL-1β is inhibited in the patient and the inflammation pathology is reduced in the patient. Identifying the patient can be by reviewing a particular patient and confirming the patient is experiencing an elevated level of IL-1β activity. The confirmation can be by determining an IL-1β level in a clinical sample from the patient, or by confirming the inflammation pathology is due to excessive IL-1β activity. The clinical indication can be, e.g., an autoinflammatory disease, a viral pneumonia, an inflammation due to an innate immune response, an inflammation response to a lipopolysaccharide, and an inflammation due to activation of macrophages.

The thione-forming disulfide can be, e.g., 6,6′-dithiodinicotinic acid (CPDS), 6,6′-dithiodinicotinic acid diethyl ester, 4-carboxypyrimidine-2-disulfide, diethyl 2,2′-dithiobis-(4-thiazole carboxylate), and 2,2′dithiobis-isonicotinic acid. The administered thione-forming disulfide dose can range from, e.g., 10 μg/kg to 5 g/kg body weight. For a human patient, the thione-forming disulfide dose can range from, e.g., 50 mg to out 800 mg per day. Administration can be by, e.g., oral administration, topical administration, or intravenous administration.

The patient can be a cancer patient experiencing excessive IL-1β activity or activated macrophage infiltration in a tumor. Alternately, there are many instances where patients or the than cancer patients can benefit from the TFD treatments. The treatment methods can administer the thione-forming disulfide in doses and schedules that reduce the IL-1β in the patient serum by at least 50% or at least 95%. The effective thione-forming disulfide can be administered at effective levels that do not cause toxic side effects to the patient, the TFDs typically being relative non-toxic. In addition, administration of TFDs can be combined with other, potentially toxic inflammation-treatment compounds, in order to reduce adverse patient effects and improve outcomes.

The methods can also include treating inflammation in an individual by administering an effective amount of a thione-forming disulfide to reduce inflammation in the individual, e.g., wherein the inflammation is associated with a disease state. The individual can be experiencing, e.g., arthritis, breast hyperplasia, psoriasis, edema, sickle cell, diabetes, multiple sclerosis, colonic polyps, or a viral pneumonia, contact with poison oak or poison ivy. In an aspect, the inflammation is mediated by arachadonic acid. The methods can include ways to provide analgesic relief in an individual by administering an effective amount of thione-forming disulfides to an individual in need of analgesic relief.

Pharmaceutical compositions for use in the methods of reducing inflammation include compositions providing effective amounts of thione-forming disulfide to a patient. In certain embodiments, the thione forming disulfide is CPDS. The compositions effectively provide the thione-forming disulfide in a dosage that reduces the IL-1β in the patient serum by at least 50%.

Further exemplary methods treat inflammation in a patient by first identifying the patient as experiencing an inflammation caused by, e.g., sickle cell disease, physical trauma, diabetes, venom, initial infections by viruses or microbes, and chemical burns. Then administering an effective dose of a thione-forming disulfide to the patient, so that IL-1β is inhibited in the patient and the inflammation pathology is reduced in the patient.

In still another aspect, the inflammation is caused by a Corona virus in the lungs of a patient. For example, in a patient identified as suffering from a viral pneumonia, a thione-forming disulfide is administered to the patient in a dose effective in reducing levels of IL-6. When IL-6 is inhibited by the TFD, the inflammation is reduced in the patient's lungs, thus limiting pathologies and mortality. In an example, the viral pneumonia is caused by a Coronavirus (e.g., SARS-COV-2), the thione-forming disulfide is CPDS, e.g., in a dose of at least 30 mg/kg. In such cases, it can be desirable to provides a concentration of at least 30 μM of CPDS in the patient's blood, e.g., oral administration, topical administration, or intravenous administration.

In a specific instance, a method of treating a COVID-19 viral pneumonia, can include identifying a patient with COVID-19 viral pneumonia, and administering CPDS to the patient in a dose of at least 30 mg/kg so that IL-6 is inhibited and inflammation (e.g., due to excessive cytokines) is reduced in the patient's lung. It can be preferred to treat COVID-19 patient when they have experienced a blood oxygen of less than 90%.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating a disease state, the method comprising:
identifying a patient with an inflammation pathology or clinical indication resulting from an overproduction of IL-1β; and,
administering an effective dose of a thione-forming disulfide to the patient;
whereby the effective dose reduces IL-1β in the patient by at least 50%.

2. The method of claim 1, wherein the clinical indication is selected from the group consisting of: infections by viruses or microbes, an autoinflammatory disease, a viral pneumonia, an inflammation due to an innate immune response, an inflammation response to a lipopolysaccharide, multiple sclerosis, sickle cell disease, physical trauma, diabetes, diabetic retinopathy, sepsis, acute respiratory distress syndrome, venom, chemical burns, and an inflammation due to activation of macrophages.

3. The method of claim 1, wherein the clinical indication is a cancer associated with overproduction of IL-1β or activated macrophage infiltration within a tumor.

4. The method of claim 1, wherein the clinical indication is selected from the group consisting of: rheumatoid arthritis, cancer, atherosclerosis, diabetes, infection by viruses and microbes, systemic lupus erythrematosus, schizophrenia, depression, major depressive disorder, Alzheimer's disease, sepsis, and acute respiratory distress syndrome.

5. The method of claim 1, wherein the thione-forming disulfide reduces the IL-1β in the patient serum by at least 95%.

6. The method of claim 1, wherein the dose provides a concentration of at least 30 μM of CPDS in the patient's blood.

7. The method of claim 1, wherein an effective amount of a thione-forming disulfide is administered to reduce inflammation.

8. A method of treating a disease state, the method comprising:
identifying a patient with an inflammation pathology or clinical indication resulting from an overproduction of IL-6; and,
administering an effective dose of a thione-forming disulfide to the patient;
whereby IL-6 is reduced in the patient by at least 50%

9. The method of claim 8, wherein the clinical indication is viral pneumonia

10. The method of claim 8, wherein the viral pneumonia is caused by a Coronavirus.

11. The method of claim 8, wherein the viral pneumonia is Covid-19.

12. The method of claim 8, wherein the thione-forming disulfide is selected from the group consisting of: 6,6′-dithiodinicotinic acid (CPDS), 6,6′-dithiodinicotinic acid diethyl ester, 4-carboxypyrimidine-2-disulfide, diethyl 2,2′-dithiobis-(4-thiazole carboxylate), and 2,2′dithiobis-isonicotinic acid.

13. The method of claim 8, wherein the thione-forming disulfide is CPDS.

14. The method of claim 8, wherein the thione-forming disulfide reduces the IL-6 in the patient serum by at least 95%.

15. The method of claim 8, wherein the dose is at least 30 mg/kg.
